SEQUENCE LISTING (6 sequences)

SEQ ID NO: 1 - DNA, 4679 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - protein, 4679 aa, adeno-associated virus 2
TTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAACTCCATCACTAGGGGTTCCTGGAGGGGTGGAGTCGTGACGTGAATTACGTCATAGGGTTAGGGAGGTCCTGTATTAGAGGTCACGTGAGTGTTTTGCGACATTTTGCGACACCATGTGGTCACGCTGGGTATTTAAGCCCGAGTGAGCACGCAGGGTCTCCATTTTGAAGCGGGAGGTTTGAACGCGCAGCCGCCATGCCGGGGTTTTACGAGATTGTGATTAAGGTCCCCAGCGACCTTGACGAGCATCTGCCCGGCATTTCTGACAGCTTTGTGAACTGGGTGGCCGAGAAGGAATGGGAGTTGCCGCCAGATTCTGACATGGATCTGAATCTGATTGAGCAGGCACCCCTGACCGTGGCCGAGAAGCTGCAGCGCGACTTTCTGACGGAATGGCGCCGTGTGAGTAAGGCCCCGGAGGCCCTTTTCTTTGTGCAATTTGAGAAGGGAGAGAGCTACTTCCACATGCACGTGCTCGTGGAAACCACCGGGGTGAAATCCATGGTTTTGGGACGTTTCCTGAGTCAGATTCGCGAAAAACTGATTCAGAGAATTTACCGCGGGATCGAGCCGACTTTGCCAAACTGGTTCGCGGTCACAAAGACCAGAAATGGCGCCGGAGGCGGGAACAAGGTGGTGGATGAGTGCTACATCCCCAATTACTTGCTCCCCAAAACCCAGCCTGAGCTCCAGTGGGCGTGGACTAATATGGAACAGTATTTAAGCGCCTGTTTGAATCTCACGGAGCGTAAACGGTTGGTGGCGCAGCATCTGACGCACGTGTCGCAGACGCAGGAGCAGAACAAAGAGAATCAGAATCCCAATTCTGATGCGCCGGTGATCAGATCAAAAACTTCAGCCAGGTACATGGAGCTGGTCGGGTGGCTCGTGGACAAGGGGATTACCTCGGAGAAGCAGTGGATCCAGGAGGACCAGGCCTCATACATCTCCTTCAATGCGGCCTCCAACTCGCGGTCCCAAATCAAGGCTGCCTTGGACAATGCGGGAAAGATTATGAGCCTGACTAAAACCGCCCCCGACTACCTGGTGGGCCAGCAGCCCGTGGAGGACATTTCCAGCAATCGGATTTATAAAATTTTGGAACTAAACGGGTACGATCCCCAATATGCGGCTTCCGTCTTTCTGGGATGGGCCACGAAAAAGTTCGGCAAGAGGAACACCATCTGGCTGTTTGGGCCTGCAACTACCGGGAAGACCAACATCGCGGAGGCCATAGCCCACACTGTGCCCTTCTACGGGTGCGTAAACTGGACCAATGAGAACTTTCCCTTCAACGACTGTGTCGACAAGATGGTGATCTGGTGGGAGGAGGGGAAGATGACCGCCAAGGTCGTGGAGTCGGCCAAAGCCATTCTCGGAGGAAGCAAGGTGCGCGTGGACCAGAAATGCAAGTCCTCGGCCCAGATAGACCCGACTCCCGTGATCGTCACCTCCAACACCAACATGTGCGCCGTGATTGACGGGAACTCAACGACCTTCGAACACCAGCAGCCGTTGCAAGACCGGATGTTCAAATTTGAACTCACCCGCCGTCTGGATCATGACTTTGGGAAGGTCACCAAGCAGGAAGTCAAAGACTTTTTCCGGTGGGCAAAGGATCACGTGGTTGAGGTGGAGCATGAATTCTACGTCAAAAAGGGTGGAGCCAAGAAAAGACCCGCCCCCAGTGACGCAGATATAAGTGAGCCCAAACGGGTGCGCGAGTCAGTTGCGCAGCCATCGACGTCAGACGCGGAAGCTTCGATCAACTACGCAGACAGGTACCAAAACAAATGTTCTCGTCACGTGGGCATGAATCTGATGCTGTTTCCCTGCAGACAATGCGAGAGAATGAATCAGAATTCAAATATCTGCTTCACTCACGGACAGAAAGACTGTTTAGAGTGCTTTCCCGTGTCAGAATCTCAACCCGTTTCTGTCGTCAAAAAGGCGTATCAGAAACTGTGCTACATTCATCATATCATGGGAAAGGTGCCAGACGCTTGCACTGCCTGCGATCTGGTCAATGTGGATTTGGATGACTGCATCTTTGAACAATAAATGATTTAAATCAGGTATGGCTGCCGATGGTTATCTTCCAGATTGGCTCGAGGACACTCTCTCTGAAGGAATAAGACAGTGGTGGAAGCTCAAACCTGGCCCACCACCACCAAAGCCCGCAGAGCGGCATAAGGACGACAGCAGGGGTCTTGTGCTTCCTGGGTACAAGTACCTCGGACCCTTCAACGGACTCGACAAGGGAGAGCCGGTCAACGAGGCAGACGCCGCGGCCCTCGAGCACGACAAAGCCTACGACCGGCAGCTCGACAGCGGAGACAACCCGTACCTCAAGTACAACCACGCCGACGCGGAGTTTCAGGAGCGCCTTAAAGAAGATACGTCTTTTGGGGGCAACCTCGGACGAGCAGTCTTCCAGGCGAAAAAGAGGGTTCTTGAACCTCTGGGCCTGGTTGAGGAACCTGTTAAGACGGCTCCGGGAAAAAAGAGGCCGGTAGAGCACTCTCCTGTGGAGCCAGACTCCTCCTCGGGAACCGGAAAGGCGGGCCAGCAGCCTGCAAGAAAAAGATTGAATTTTGGTCAGACTGGAGACGCAGACTCAGTACCTGACCCCCAGCCTCTCGGACAGCCACCAGCAGCCCCCTCTGGTCTGGGAACTAATACGATGGCTACAGGCAGTGGCGCACCAATGGCAGACAATAACGAGGGCGCCGACGGAGTGGGTAATTCCTCGGGAAATTGGCATTGCGATTCCACATGGATGGGCGACAGAGTCATCACCACCAGCACCCGAACCTGGGCCCTGCCCACCTACAACAACCACCTCTACAAACAAATTTCCAGCCAATCAGGAGCCTCGAACGACAATCACTACTTTGGCTACAGCACCCCTTGGGGGTATTTTGACTTCAACAGATTCCACTGCCACTTTTCACCACGTGACTGGCAAAGACTCATCAACAACAACTGGGGATTCCGACCCAAGAGACTCAACTTCAAGCTCTTTAACATTCAAGTCAAAGAGGTCACGCAGAATGACGGTACGACGACGATTGCCAATAACCTTACCAGCACGGTTCAGGTGTTTACTGACTCGGAGTACCAGCTCCCGTACGTCCTCGGCTCGGCGCATCAAGGATGCCTCCCGCCGTTCCCAGCAGACGTCTTCATGGTGCCACAGTATGGATACCTCACCCTGAACAACGGGAGTCAGGCAGTAGGACGCTCTTCATTTTACTGCCTGGAGTACTTTCCTTCTCAGATGCTGCGTACCGGAAACAACTTTACCTTCAGCTACACTTTTGAGGACGTTCCTTTCCACAGCAGCTACGCTCACAGCCAGAGTCTGGACCGTCTCATGAATCCTCTCATCGACCAGTACCTGTATTACTTGAGCAGAACAAACACTCCAAGTGGAACCACCACGCAGTCAAGGCTTCAGTTTTCTCAGGCCGGAGCGAGTGACATTCGGGACCAGTCTAGGAACTGGCTTCCTGGACCCTGTTACCGCCAGCAGCGAGTATCAAAGACATCTGCGGATAACAACAACAGTGAATACTCGTGGACTGGAGCTACCAAGTACCACCTCAATGGCAGAGACTCTCTGGTGAATCCGGGCCCGGCCATGGCAAGCCACAAGGACGATGAAGAAAAGTTTTTTCCTCAGAGCGGGGTTCTCATCTTTGGGAAGCAAGGCTCAGAGAAAACAAATGTGGACATTGAAAAGGTCATGATTACAGACGAAGAGGAAATCAGGACAACCAATCCCGTGGCTACGGAGCAGTATGGTTCTGTATCTACCAACCTCCAGAGAGGCAACAGACAAGCAGCTACCGCAGATGTCAACACACAAGGCGTTCTTCCAGGCATGGTCTGGCAGGACAGAGATGTGTACCTTCAGGGGCCCATCTGGGCAAAGATTCCACACACGGACGGACATTTTCACCCCTCTCCCCTCATGGGTGGATTCGGACTTAAACACCCTCCTCCACAGATTCTCATCAAGAACACCCCGGTACCTGCGAATCCTTCGACCACCTTCAGTGCGGCAAAGTTTGCTTCCTTCATCACACAGTACTCCACGGGACAGGTCAGCGTGGAGATCGAGTGGGAGCTGCAGAAGGAAAACAGCAAACGCTGGAATCCCGAAATTCAGTACACTTCCAACTACAACAAGTCTGTTAATGTGGACTTTACTGTGGACACTAATGGCGTGTATTCAGAGCCTCGCCCCATTGGCACCAGATACCTGACTCGTAATCTGTAATTGCTTGTTAATCAATAAACCGTTTAATTCGTTTCAGTTGAACTTTGGTCTCTGCGTATTTCTTTCTTATCTAGTTTCCATGGCTACGTAGATAAGTAGCATGGCGGGTTAATCATTAACTACAAGGAACCCCTAGTGATGGAGTTGGCCACTCCCTCTCTGCGCGCTCGCTCGCTCACTGAGGCCGGGCGACCAAAGGTCGCCCGACGCCCGGGCTTTGCCCGGGCGGCCTCAGTGAGCGAGCGAGCGCGCAGAGAGGGAGTGGCCAA

SEQ ID NO: 3 - DNA, 42 bp, adeno-associated virus 5
caataaatgatttaaatcaggtatgtcttttgttgatcaccc

SEQ ID NO: 4 - DNA, 31 bp, adeno-associated virus 5
gatgttgtaagctgttattcattgaatgacc

SEQ ID NO: 5 - DNA, 42 bp, adeno-associated virus 2
gggtgatcaacaaaagacatacctgatttaaatcatttattg

SEQ ID NO: 6 - DNA, 29 bp, adeno-associated virus 2
gattgagcaggcacccctgaccgtggccg

(19) United States
(12) Patent Application Publication
(10) Pub. No.: US 2002/0045264 A1
(43) Pub. Date: Apr. 18, 2002

(54) Production of chimeric capsid vectors

(76) Inventors: Matthew J. During, Philadelphia, PA (US); Weidong Xiao, Jenkintown, PA (US)

Correspondence Address: NUTTER MCCLENNEN & FISH LLP, ONE INTERNATIONAL PLACE, BOSTON, MA, 02110, US

(21) Appl. No.: 09/804,898

(22) Filed: Mar. 13, 2001

Related U.S. Application Data
(60) Provisional application No. 60/189,110, filed on Mar. 14, 2000.

(51) Int. Cl. C12N 15/861; C12N 7/01

(52) U.S. Cl. 435/456; 435/235.1; 435/320.1

(57) ABSTRACT

The present invention related to methods and compositions comprising recombinant vectors comprising chimeric capsids. The chimeric capsids confer an altered tropism that permits selective targeting of desired cells.

This application claims priority to U.S. Provisional Patent Application No. 60/189,110, filed Mar. 14, 2000.

### BACKGROUND OF THE INVENTION

The technical field of this invention is recombinant viral vectors and, in particular, recombinant viral vectors with a chimeric capsid derived from at least two parvoviruses, or derived from at least one parvovirus and at least one virus other then a parvovirus.

Parvoviridae are small non-enveloped viruses containing single-stranded linear DNA genomes of 4 to 6 kb in length. Adeno-associated virus (AAV) is a member of the parvoviridae family. The AAV genome contains major open reading frames coding for the Rep (replication) and Cap (capsid) proteins. Flanking the AAV coding regions are two nucleotide inverted terminal repeat (ITR) sequences which contain palindromic sequences that can fold over to form hairpin structures that function as primers during initiation of DNA replication. In addition to their role in DNA replication, the ITR sequences have been shown to be necessary for viral integration, rescue from the host genome and encapsidation of viral nucleic acid into mature virions (Muzyczka, (1992) Curr. Top. Micro. Immunol. 158:97-129).

The capsids have icosahedral symmetry and are about 20-24 nm in diameter. They are composed of three viral proteins (VP1, VP2, and VP3, which are approximately 87, 73 and 61 Kd, respectively) (Muzyczka supra). VP3 represents 90% of the total virion protein; VP2 and VP1 account for approximately 5 % each.

AAV can assume two pathways upon infection of a host cell. In the presence of helper virus, AAV will enter the lytic pathway where the viral genome is transcribed, replicated, and encapsidated into newly formed viral particles. In the absence of helper virus function, the AAV genome becomes integrated as a provirus into a specific region of the host cell genome, through recombination between the AAV ITRs and host cell sequences. Specific targeting of AAV viral DNA occurs at the long arm of human chromosome 19 (Kotin et al., (1990) Proc. Natl. Acad. Sci. USA 87:2211-2215; Samulski et al., (1991) EMBO J. 10:3941-3950). This particular feature of AAV reduces the likelihood of insertional mutagenesis resulting from random integration of viral vector DNA into the coding region of a host gene.

The AAV viral particle uses cellular receptors to attach to and infect a cell. Recently identified receptors include a heparan sulfate proteoglycan receptor as the primary receptor, and either the fibroblast growth factor (FGF), or the integrin aVb5, as secondary receptors. Following attachment to the cell, the viral particle undergoes receptor-mediated internalization into clathrin-coated endocytic vesicles of the cell.

The AAV vector has properties that make it unique for gene therapy, for example, AAV is not associated with any known diseases and is generally non-pathogenic. In addition, AAV integrates into the host chromosome in a site-specific manner (See e.g., Kotin et al., (1990) Proc. Natl. Acad. Sci. 87: 2211-2215 and Samulski et al., (1991) EMBO J. 10: 3941-3950).

Although the AAV virus vectors provide a suitable means for gene delivery to a target cell, they may often display a limited tropism for particular cell types. To date, attempts to alter the tropism of AAV vectors have involved introducing a peptide ligand into the capsid coat. For example, Girod et al. introduced a 14 amino acid peptide containing the RDG motif of the laminin fragment P1 into a capsid region of the AAV-2 serotype to alter tropism (Girod et al. (1999) Nature Med. 5: 1052-1056). Zavada et al. altered the tropism of an AAV vector by the addition of viral glycoproteins (Zavada et al. (1982) J. Gen. Virol. 63: 15-24). Others have added single chain fragments of variable regions of a monoclonal antibody against CD34 to the N-terminus of the VP2 capsid (Yang et al. (1998) Hum. Gene. Ther. 9: 1929-1937). The major limitation with these approaches is that they require additional steps that covalently link large molecules, such as receptor ligands and antibodies to the virus. This adds to the size of the virus as well as the cost of production. Furthermore, the targeted particles are not homogenous in structure, which may effect the efficiency of gene transfer. Therefore, a need exists to generate viral vectors with an altered tropism that is efficient for gene transfer.

### SUMMARY OF THE INVENTION

The invention is based on the discovery that a recombinant vector with a chimeric capsid can be produced. The recombinant vector has at least one non-native amino acid sequence derived from a capsid protein from another member of the parvovirus family, and also contains a packaging sequence in the genome that can be derived from the wild type parvovirus or can be derived from another family member. Accordingly, the invention provides modular approach to producing a recombinant vector comprising a chimeric capsid that is both versatile and flexible. The resulting recombinant vector has a modified tropism that allows the recombinant vector to interact with a cell surface molecule with a higher affinity than a recombinant vector with a wild type capsid. Thus, the chimeric capsid allows targeting of cells that a wild type capsid would not normally target. The modular approach involves producing a recombinant vector that comprises at least two different components derived from different viruses. The two different components can be capsid protein components, inverted terminal repeat sequences or any combinations thereof.

Accordingly, in one aspect, the invention features recombinant viral vector comprising:

a chimeric capsid having at least one non-native amino acid sequence, wherein the non-native amino acid sequence is derived from a capsid protein domain of a parvovirus, a virus, or a combination thereof, and wherein the chimeric capsid is capable of binding to an attachment site present on a cell surface; and

a transgene flanked 5′ and 3′ by inverted terminal repeat sequences, wherein the inverted terminal repeat sequences are derived from a parvovirus, a virus, or a combination thereof, and wherein at least one inverted terminal repeat sequence comprises a packaging signal that allows assembly of the chimeric capsid.

The chimeric capsid has an modified tropism that permits binding of the viral vector to an attachment site on a cell surface with higher affinity than a corresponding viral vector with a wild type capsid. Alternatively, the modified tropism can prevent the chimeric capsid from binding to an attachment site on a cell surface.

In one embodiment, the parvovirus selected from the group consisting of AAV-1, AAV-2, AAV-3, AAV-4, AAV-5 and AAV-6. The parvovirus comprises a capsid protein with viral protein domains selected from the group consisting of VP1, VP2 and VP3. In one embodiment, the non-native amino acid sequence is a combination of amino acid sequences derived from one or more parvoviruses selected from the group consisting of AAV-1, AAV-2, AAV-3, AAV-4, AAV-5 and AAV-6. In a preferred embodiment, the non-native amino acid sequence is a combination of an amino acid sequence derived from AAV-2 and an amino acid sequence derived from AAV-5.

In one embodiment, the non-native amino acid sequence is derived from a virus, for example, a virus is selected from the group consisting of herpesvirus, adenovirus, lentivirus, retrovirus, Epstein-Barr virus and vaccinia virus.

In another embodiment, the non-native amino acid sequence is a combination of at least one amino acid sequence derived from a parvovirus selected from the group consisting of AAV-1, AAV-2, AAV-3, AAV-4, AAV-5 and AAV-6, and at least one amino acid sequence derived from a virus selected from the group consisting of herpesvirus, adenovirus, lentivirus, retrovirus, Epstein-Barr virus and vaccinia virus.

In one embodiment, the inverted terminal repeat sequences are each derived from a parvovirus selected from the group consisting of AAV-1, AAV-2, AAV-3, AAV-4, AAV-5 and AAV-6. In another embodiment, the inverted terminal repeat sequences are each derived from a viruses selected from the group consisting of herpesvirus, adenovirus, lentivirus, retrovirus, Epstein-Barr virus and vaccinia virus. In yet another embodiment, the inverted terminal repeat sequences are a combination of at least one inverted terminal repeat sequence derived from a parvovirus selected from the group consisting of AAV-1, AAV-2, AAV-3, AAV-4, AAV-5 and AAV-6, and at least one inverted terminal repeat sequence derived from a virus selected from the group consisting of herpesvirus, adenovirus, lentivirus, retrovirus, Epstein-Barr virus and vaccinia virus.

In one embodiment, the transgene is selected from the group consisting of an RNA molecule, a DNA molecule, and a synthetic DNA molecule.

In another aspect, the invention features a recombinant AAV-2 vector comprising:

a chimeric capsid having at least one native AAV-2 amino acid sequence and at least one non-native amino acid sequence derived from a parvovirus other than AAV-2, wherein the chimeric capsid is capable of binding to an attachment site present on a cell surface; and

a transgene flanked 5′ and 3′ by a first inverted terminal repeat sequences derived from AAV-2 and a second inverted terminal repeat sequence derived from a parvovirus.

In one embodiment, the amino acid sequence derived from AAV-2 comprises a viral protein domain selected from the group consisting of VP1, VP2 and VP3. In one embodiment, the non-native amino acid sequence is derived from a parvovirus selected from the group consisting of AAV-1, AAV-3, AAV-5 and AAV-6. The non-native amino acid sequence of the parvovirus comprises a viral protein domain selected from the group consisting of VP1, VP2 and VP3. In a preferred embodiment, the chimeric capsid comprises a native amino acid sequence from the VP1 domain of AAV-2 and wherein the non-native amino acid sequence comprises a VP2 domain of AAV-5 and a VP3 domain of AAV-5. In one embodiment, the second inverted terminal repeat sequence derived from a parvovirus selected from the group consisting of AAV-1, AAV-2, AAV-3, AAV-4, AAV-5 and AAV-6.

In another aspect, the invention features a recombinant AAV-2 vector comprising:

a chimeric capsid having at least one native AAV-2 amino acid sequence and at least one non-native amino acid sequence derived from a virus, wherein the chimeric capsid is capable of binding to an attachment site present on a cell surface; and

a transgene flanked 5′ and 3′ by a first inverted terminal repeat sequence derived from AAV-2 and a second inverted terminal repeat sequence derived from a parvovirus.

In one embodiment, the non-native amino acid sequence is derived from a virus selected from the group consisting of herpesvirus, adenovirus, lentivirus, retrovirus, Epstein-Barr virus and vaccinia virus. In one embodiment, the second inverted terminal repeat sequence is derived from a parvovirus selected from the group consisting of AAV-1, AAV-3, AAV-4, AAV-5 and AAV-6.

In one aspect, the invention features a recombinant AAV-2 vector comprising:

a chimeric capsid having at least one native AAV-2 amino acid sequence and at least one non-native amino acid sequence derived from a virus, wherein the chimeric capsid is capable of binding to an attachment site present on a cell surface; and

a transgene flanked by a first inverted terminal repeat sequence from AAV-2 and a second inverted terminal repeat sequence from a virus.

In one embodiment, the second terminal repeat sequence is derived from a virus selected from the group consisting of herpesvirus, adenovirus, lentivirus, retrovirus, Epstein-Barr virus and vaccinia virus.

In another aspect, the invention features a chimeric capsid vehicle comprising a native AAV-2 amino acid sequence and at least one non-native amino acid sequence derived from a capsid protein of a parvovirus other than AAV-2, covalently linked to a transgene.

In another aspect, the invention features a chimeric capsid vehicle comprising a native AAV-2 amino acid sequence and at least one non-native amino acid derived from a capsid protein of a virus, covalently linked to a transgene.

In another aspect, the invention features a method for modifying the tropism of a recombinant AAV-2 vector comprising:

replacing at least a portion of a native amino acid sequence of an AAV-2 capsid protein with a non-native amino acid sequence derived from a capsid protein of a parvovirus other than AAV-2; and

combining the capsid proteins under conditions for assembly, to thereby modify the tropism of an AAV-2 vector.

In another aspect, the invention features a method for modifying the tropism comprising:

replacing at least a portion of a native amino acid sequence of an AAV-2 capsid protein with a non-native amino acid sequence derived from a capsid protein of a virus; and

combining the capsid protein under conditions for assembly, to thereby modify the tropism of an AAV-2 vector.

In another aspect, the invention features a method for improving gene therapy in a subject with a disorder comprising:

administering a therapeutically effective amount of a recombinant vector comprising a transgene and a chimeric capsid capable of binding to an attachment site present on a cell surface;

targeting a cell that a recombinant vector with a chimeric capsid can bind to with a higher affinity than the corresponding viral vector with a wild type capsid; and

expressing the transgene in a subject at a level sufficient to ameliorate the disorder thereby improving gene therapy.

In one embodiment, the recombinant vector comprising a chimeric capsid comprises at least one amino acid sequence derived from a viral protein domain of a first parvovirus and at least one amino acid sequence derived from a viral protein domain or a second parvovirus. The first parvovirus is selected from the group consisting of AAV-1, AAV-2, AAV-3, AAV-4, AAV-5 and AAV-6. The second parvovirus is selected from the group consisting of AAV-1, AAV-2, AAV-3, AAV-4, AAV-5 and AAV-6.

In another embodiment, the recombinant vector comprising a chimeric capsid comprises at least one amino acid sequence derived from a parvovirus and at least one amino acid sequence derived from a virus. The parvovirus is selected from the group consisting of AAV-1, AAV-2, AAV-3, AAV-4, AAV-5 and AAV-6. The virus is selected from the group consisting of herpesvirus, adenovirus, lentivirus, retrovirus, Epstein-Barr virus and vaccinia virus.

In another embodiment, the recombinant vector comprising a chimeric capsid comprises at least one amino acid sequence derived from AAV-2 and at least one amino acid sequence derived from a parvovirus. The parvovirus is selected from the group consisting of AAV-1, AAV-3, AAV-5 and AAV-6.

In another embodiment, the recombinant vector comprising a chimeric capsid comprises at least one amino acid sequence derived from AAV-2 and at least one amino acid sequence derived from a virus. The virus is selected from the group consisting of herpesvirus, adenovirus, lentivirus, retrovirus, Epstein-Barr virus and vaccinia virus.

In another aspect, the invention features a method for increasing the efficiency of entry into a cell using a recombinant viral vector with a chimeric capsid comprising:

producing a chimeric capsid encapsidating a viral vector, wherein the chimeric capsid has a modified tropism; and

contacting a cell with the recombinant viral vector having a chimeric capsid such that the chimeric capsid binds to an attachment site on the cell surface and permits the vector to enter the cell more efficiently that a viral vector comprising a wild type capsid.

In another aspect, the invention features a method of making a recombinant particle with a chimeric capsid comprising:

providing a first construct comprising a transgene flanked 5′ and 3′ with inverted terminal repeat sequences, wherein at least one invented terminal repeat sequence comprises a packaging signal, and a second construct comprising a nucleic acid sequence encoding a chimeric capsid; and

contacting a population of cells with the first and second constructs, such that the population of cells allows assembly of a recombinant particle, to thereby produce a recombinant particle with a chimeric capsid.

The another aspect, the invention also features isolated nucleic acid sequences encoding the chimeric capsids, cells, and pharmaceutical composition comprising the recombinant vectors.

### DETAILED DESCRIPTION

The present invention is based on the discovery that a recombinant adeno-associated virus (AAV) vector containing a chimeric capsid can be packaged efficiently producing recombinant vector with a chimeric capsid that has a modified tropism. The modified tropism allows the recombinant vector to bind to attachment sites on target cells with a higher affinity than a recombinant vector with wild type capsid.

So that the invention is more clearly understood, the following terms are defined:

The term “gene transfer” or “gene delivery” as used herein refers to methods or systems for reliably inserting foreign DNA into host cells. Such methods can result in transient expression of non-integrated transferred DNA, extra-chromosomal replication and expression of transferred replicons (e.g., episomes), or integration of transferred genetic material into the genomic DNA of host cells. Gene transfer provides a unique approach for the treatment of acquired and inherited diseases. A number of systems have been developed for gene transfer into mammalian cells. (See, e.g., U.S. Pat. No. 5,399,346.)

The term “vector” as used herein refers to any genetic element, such as a plasmid, phage, transposon, cosmid, chromosome, virus, virion, and the like, which is capable of replication when associated with the proper control elements and which can transfer gene sequences into cells. Thus, the term includes cloning and expression vehicles, as well as viral vectors.

The term “AAV vector” as used herein refers to a vector derived from an adeno-associated virus serotype, including but not limited to, AAV-1, AAV-2, AAV-3, AAV-4, AAV-5, AAVX7, and the like. AAV vectors can have one or more of the AAV wild-type genes deleted in whole or part, preferably the rep and/or cap genes, but retain functional flanking Inverted Terminal Repeat (ITR) sequences. Functional ITR sequences permit the rescue, replication and packaging of the AAV virion. Thus, an AAV vector is defined herein to include at least those sequences required for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The term “transgene”, as used herein, is intended to refer to a gene sequence and are nucleic acid molecules. Such transgenes, or gene sequences, may be derived form a variety of sources including DNA, cDNA, synthetic DNA, and RNA. Such transgenes may comprise genomic DNA which may or may not include naturally occurring introns. Moreover, such genomic DNA may be obtained in association with promoter regions or poly A sequences. The transgenes of the present invention are preferably cDNA. Genomic or cDNA may be obtained by means well known in the art. A transgene which may be any gene sequence whose expression produces a gene product that is to be expressed in a cell. The gene product may affect the physiology of the host cell. Alternatively the transgene may be a selectable marker gene or reporter gene. The transgene can be operably linked to a promoter or other regulatory sequence sufficient to direct transcription of the transgene. Suitable promoters include, for example, as human CMV IEI promoter or an SV40 promoter.

The term “regulatory sequence” is art-recognized and intended to include control elements such as promoters, enhancers and other expression control elements (e.g., polyadenylation signals), transcription termination sequences, upstream regulatory domains, origins of replication, internal ribosome entry sites (“IRES”), enhancers, enhancer sequences, post-regulatory sequences and the like, which collectively provide for the replication, transcription and translation of a coding sequence in a recipient cell. Not all of these regulatory sequences need always be present so long as the selected coding sequence is capable of being replicated, transcribed and translated in an appropriate host cell. Such regulatory sequences are known to those skilled in the art and are described in Goeddel, Gene Expression Technology: Methods in Enzymology 185, Academic Press, San Diego, Calif. (1990). It should be understood that the design of the viral vector may depend on such factors as the choice of the host cell to be transfected and/or the amount of protein to be expressed.

The term “promoter” is used herein refers to the art recognized use of the term of a nucleotide region comprising a regulatory sequence, wherein the regulatory sequence is derived from a gene which is capable of binding RNA polymerase and initiating transcription of a downstream (3′-direction) coding sequence.

The term “operably linked” as used herein refers to an arrangement of elements wherein the components are configured so as to perform their usual function. Thus, control elements operably linked to a coding sequence are capable of effecting the expression of the coding sequence. The control elements need not be contiguous with the coding sequence, so long as they function to direct the expression of the coding sequence. For example, intervening untranslated yet transcribed can be present between a promoter sequence and the coding sequence and the promoter sequence can still be considered “operably linked” to the coding sequence.

The terms “5′”, “3′”, “upstream” or “downstream” are art recognized terms that describe the relative position of nucleotide sequences in a particular nucleic acid molecule relative to another sequence.

The term “recombinant particle,” as used herein refers to an infectious, replication-defective virus composed of a viral coat, encapsidating a transgene which is flanked on both sides by viral ITRs. For example, the recombinant particle can be a recombinant AAV particle. A recombinant AAV particle can be produced in a suitable host cell which has had an AAV vector, AAV helper functions and/or accessory functions introduced therein. In this manner, the host cell is rendered capable of encoding AAV capsid proteins that are required for packaging the AAV vector (containing a transgene) into recombinant particles for subsequent gene delivery.

The term “AAV rep coding region” as used herein refers to the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other exogenous) promoters. The Rep expression products are collectively required for replicating the AAV genome. For a description of the AAV rep coding region, see, e.g., Muzyczka (1992) Current Topics in Microbiol. and Immunol. 158:97-129; and Kotin (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

The term “AAV cap coding region” as used herein refers to the art-recognized region of the AAV genome which encodes the capsid proteins VP1, VP2, and VP3, or functional homologues thereof. These cap expression products supply the packaging functions which are collectively required for packaging the viral genome. For a description of the AAV cap coding region, See, e.g., Muzyczka (Supra).

The term “chimeric capsid” as used herein refers to a viral protein coat with one or more non-native amino acid sequences. The chimeric capsid can comprise a combination of amino acid sequences from the same family. For example, a chimeric capsid comprising the VP1 domain of AAV-2, in combination with the VP2 and VP3 domains of AAV-5. The skilled artisan will appreciate that the chimeric capsid can be any combination of viral protein domains from the parvovirus family member such as, AAV-1, AAV-2, AAV-3, AAV-4, AAV-5 and AAV-6. The invention however, excludes a chimeric capsid with the combination of a viral protein domain of AAV-2 and a viral protein domain of AAV-4. The term chimeric capsid also refers to a viral protein coat with at least one non-native amino acid sequence from a virus, such as herpesvirus, adenovirus, lentivirus, retrovirus, Epstein-Barr virus and vaccinia virus, and the like.

A “fragment” or “portion” of a nucleic acid encoding a capsid protein is defined as a nucleotide sequence having fewer nucleotides than the nucleotide sequence encoding the entire amino acid sequence of the capsid protein, such as VP1, VP2 or VP3. A fragment or portion of a nucleic acid molecule is about 20 nucleotides, preferably about 30 nucleotides, more preferably about 40 nucleotides, even more preferably about 50 nucleotides in length. Also within the scope of the invention are nucleic acid fragments which are about 60, 70, 80, 90, 100 or more nucleotides in length. Preferred fragments or portions include nucleotide sequences encode a polypeptide that alters the tropism of the chimeric capsid. The term fragment or portion also refers to an amino acid sequence of the capsid protein that has fewer amino acids than the entire sequence of the viral protein domains VP1, VP2 and VP3. The fragment is about 10 amino acids, more preferably about 20, 30, 40, 50, 60, 70, 80, 90, 100, 120, 140, 160, 180 and 200 or more amino acids in length.

The term “transfection” is used herein refers to the uptake of an exogenous nucleic acid molecule by a cell. A cell has been “transfected” when exogenous nucleic acid has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acid molecules into suitable host cells. The term refers to both stable and transient uptake of the nucleic acid molecule.

The term “coding sequence” or a sequence which “encodes” or sequence “encoding” a particular protein, as used herein refers to a nucleic acid molecule which is transcribed (in the case of DNA) and translated (in the case of messenger mRNA) into a polypeptide in vitro or in vivo when placed under the control of appropriate regulatory sequences. A gene can include, but is not limited to, cDNA from prokaryotic or eukaryotic mRNA, genomic DNA sequences from prokaryotic or eukaryotic DNA, and even synthetic DNA sequences.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The terms “polypeptide” and “protein” are used interchangeably herein and refer to a polymer of amino acids and includes full-length proteins and fragments thereof. As will be appreciated by those skilled in the art, the invention also includes nucleic acids that encode those polypeptides having slight variations in amino acid sequences or other properties from a known amino acid sequence. Amino acid substitutions can be selected by known parameters to be neutral and can be introduced into the nucleic acid sequence encoding it by standard methods such as induced point, deletion, insertion and substitution mutants. Minor changes in amino acid sequence are generally preferred, such as conservative amino acid replacements, small internal deletions or insertions, and additions or deletions at the ends of the molecules. These modifications can result in changes in the amino acid sequence, provide silent mutations, modify a restriction site, or provide other specific mutations. Additionally, they can result in a beneficial change to the encoded protein.

The term “homology” or “identity” as used herein refers to the percentage of likeness between nucleic acid molecules. To determine the homology or percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. (48):444-453) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In another example, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In yet another example, the percent identity between two amino acid or nucleotide sequences is determined using the algorithm of E. Meyers and W. Miller (CABIOS, 4:11-17 (1989)) which has been incorporated into the ALIGN program (version 2.0), using a PAM 120 weight residue table, a gap length penalty of 12 and a gap penalty.

Further details of the invention are described in the following sections:

I Recombinant Vectors Comprising Chimeric Capsid

The invention features a method of producing recombinant vectors comprising a chimeric capsid. Recombinant vectors can be constructed using known techniques to provide operatively linked components of control elements including a transcriptional initiation region, a transgene, and a transcriptional termination region. The control elements are selected to be functional in the targeted cell. The resulting construct which contains the operatively linked components can be flanked at the 5′ and 3′ region with functional parvoviral ITR sequences.

In one embodiment, the invention features a recombinant viral vector comprising a chimeric capsid having at least one non-native amino acid sequence, wherein the non-native amino acid sequence is derived from a capsid protein domain of a parvovirus, a virus, or a combination thereof, and wherein the chimeric capsid is capable of binding to an attachment site present on a cell surface; and a transgene flanked 5′ and 3′ by inverted terminal repeat sequences, wherein the inverted terminal repeat sequences are derived from a parvovirus, a virus, or a combination thereof, and wherein at least one inverted terminal repeat sequence comprises a packaging signal that allows assembly of the chimeric capsid.

The parvovirus family includes adeno-associated viruses. Examples of adeno-associated virus serotypes include, but are not limited to, AAV-1 (Xiao et al. (1999), J. Virol., 73: 3994-4003, GenBank Accession No. AF063497), AAV-2 (Ruffing et al. (1994) J. Gen. Virol., 75: 3385-3392, GenBank Accession No. AF043303), AAV-3 (Muramatsu et al. (1996) Virology 221: 208-217, GenBank Accession No. U48704; Rutledge et al. (1998) J. Virol., 72: 309-319, GenBank Accession No. AF028705), AAV-4 (Chiorini et al. (1997), J. Virol., 71: 6823-6833, GenBank Accession No. U89790), AAV-5 (Bantel et al., (1999), J. Virol. 73: 939-947 GenBank Accession No. Y18065) and AAV-6 (Rutledge et al. (1998), J. Virol., 72: 309-319, GenBank Accession No. AF028704). The sequences of the capsid genes for such serotypes is reported in Srivastava et al., (1983) J. Virol. 45:555-564; Muzyczka (1992) Curr. Top. Micro Immunol. 158:97-129, and Ruffing et al. (1992) J. Virol. 66:6922-6930. Each serotype of AAV has a different cellular tropism and bind to different cell surface proteins. Some parvovirus family members are useful for transduction of particular cell types, but less useful for transduction of other cells.

A particularly preferred parvovirus is the adeno-associated virus (AAV-2). AAV-2 has a broad host range and until recently, all human cells were thought to be infectable. However, certain cells of the central nervous system are inaccessible with AAV-2. For example, AAV-2 has poor tropism for human myeloid stem cells, or cells form the lymphocyte lineage. AAV-2 is not associated with any disease, therefore making it safe for gene transfer applications (Cukor et al. (1984), The Parvoviruses, Ed. K. I. Bems, Plenum, N. Y., 33-36; Ostrove et al. (1981), Virology 113: 521). AAV-2 integrates into the host genome upon infection so that transgene can be expressed indefinitely (Kotin et al. (1990), Proc. Natl. Acad. Sci. USA 87: 221; Samulski et al.(1991), EMBO J. 10: 3941). Integration of AAV-2 into the cellular genome is independent of cell replication which is particularly important since AAV can thus transfer genes into quiescent cells (Lebkowski et al. (1988), Mol. Cell. Biol. 8: 3988).

Accordingly, in one embodiment, the invention features a recombinant AAV-2 vector comprising a chimeric capsid having at least one native AAV-2 amino acid sequence and at least one non-native amino acid sequence derived from a parvovirus other than AAV-2, wherein the chimeric capsid is capable of binding to an attachment site present on a cell surface; and a transgene flanked 5′ and 3′ by a first inverted terminal repeat sequences derived from AAV-2 and a second inverted terminal repeat sequence derived from a parvovirus.

In one embodiment, the chimeric capsids of the recombinant vectors are produced by “complete substitutions”, this term as used herein refers to replacing the entire capsid viral protein domain of the host with a non-native amino acid sequence. For example, a recombinant AAV-2 vector in which the amino acid sequence of the VP1 domain of AAV-2 is retained, but the entire amino acid sequence of the VP2 and VP3 domain of AAV-2 is replaced with the entire amino acid sequence of the VP2 domain from another parvovirus, such as AAV-5.

In another embodiment, the chimeric capsids of the recombinant vectors are produced by “patch substitution” this term as used herein refers to replacing a fragment of the capsid viral protein domain of the host with a fragment of non-native amino acid sequence from another parvovirus. For example, a recombinant AAV-2 vector in which a fragment of the amino acid sequence of the VP1 domain of AAV-2 is replaced with a corresponding fragment of a non-native amino acid sequence from AAV-5. The non-native amino acid sequence preferably comprises a determinant that alters the tropism of the capsid. The altered tropism can allow the chimeric capsid to bind to an attachment site on cell surface with a higher affinity than a wild type capsid. The modified tropism of the chimeric capsid allows a wider range of host cells to be targeted. The infective properties of such a particle can be improved above those of a recombinant vector containing a wild type capsid. Alternatively, the altered tropism can prevent the chimeric capsid from binding to an attachment site on a cell surface. This provides for a method of selecting cell types for specific targeting of a transgene, while excluding expression of the transgene where it is not wanted.

In one embodiment, the invention features recombinant vectors with a chimeric capsid where the chimeric capsid comprises fragments of the entire AAV-2 capsid protein, VP1, VP2, or VP3 sequences. The fragments can be an amino acid sequence comprising about 10 amino acids, more preferably about 20, 30, 40, 50, 60, 70, 80, 90, 100, 120, 140, 160, 180 and 200 or more amino acids in length.

Additionally, modifications can be made to the nucleic acid molecule encoding the capsid protein or fragment thereof, such that modifications to the nucleotide sequences that encode a capsid protein produce a capsid protein with a modified amino acid sequence. Such means of generating modification to a sequence are standard in the art (See e.g., Sambrook J., Fritsch E. F., Maniatis T.: Molecular cloning: a laboratory manual. Cold Spring Harbor, N. Y., Cold Spring Harbor Laboratory, 1989) and can be performed.

Also within the scope of the invention are AAV-2 recombinant vectors with a chimeric capsid comprising VP1, VP2, VP3 proteins that can have at least 60% homology to the polypeptide encoded by nucleotides at position 2202 to nucleotide at position 4412 set forth in SEQ ID NO: 1. The full length nucleotide sequence set forth in SEQ ID NO: 1 is the entire genome of AAV-2 and encodes the amino acid sequence set forth in SEQ ID NO: 2. The capsid protein can have about 70% homology, about 75% homology, about 80% homology, about 85% homology, about 90% homology, about 95% homology, about 99% homology to the polypeptide encoded by nucleotides at position 2202 to nucleotide at position 4412 set forth in SEQ ID NO: 1.

Examples of attachment sites present on a surface cell types that can be targeted by the recombinant vector with the chimeric capsid include, but are not limited to heparin and chondroitin sulfate moities found on glycosaminoglycans, sialic acid moieties found on mucins, glycoproteins, gangliosides, MHC class I glycoproteins, common carbohydrate components found in the cell membrane glycoproteins including mannose, N-acetyl-galactosamine, fucose, galactose and the like.

Examples of a suitable transgene used in the recombinant vector of the invention include gene sequences for amyloid polyneuropathy, Alzheimer's Disease, Duchenne's muscular dystrophy, ALS, Parkinson's Disease and brain tumors. The transgene may also be a selectable marker gene which is any gene sequence capable of expressing a protein whose presence permits selective propagation of a cell which contains it. Examples of selectable markers include gene sequence capable of conferring host resistance to antibiotics (such as ampicillin, tetracycline, kanamycin, etc.), amino acid analogs, or permitting growth of bacteria on additional carbon sources or under otherwise impermissible culturing conditions.

The skilled artisan can appreciate that regulatory sequences to control expression of the transgene can often be provided from commonly used promoters derived from viruses such as, polyoma, Adenovirus 2, lentivirus, retrovirus, and Simian Virus 40. Use of viral regulatory elements to direct expression of the transgene can allow for high level constitutive expression of the protein in a variety of host cells. Ubiquitously expressing promoters can also be used include, for example, the early lentivirus, retrovirus, promoter Boshart et al. (1985) Cell 41:521-530, herpesvirus thymidine kinase (HSV-TK) promoter (McKnight et al. (1984) Cell 37: 253-262), β-actin promoters (e.g., the human β-actin promoter as described by Ng et al. (1985) Mol. Cell Biol. 5: 2720-2732) and colony stimulating factor-1 (CSF-1) promoter (Ladner et al., (1987) EMBO J. 6: 2693-2698).

Alternatively, the regulatory sequences can direct expression of the transgene preferentially in a particular cell type, i.e., tissue-specific regulatory elements can be used. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). The promoter can be any desired promoter, selected based on the level of expression required of the transgene operably linked to the promoter and the cell type in which the vector is used. In one embodiment, the promoter is an AAV-2 promoter selected from the group consisting of p5, p19 and p40. In a preferred embodiment, the promoter is an AAV-2 p5 promoter.

The recombinant vector comprising the chimeric capsid can be packaged into a particle using a transgene flanked by the same parvovirus ITR sequences e.g., AAV-2 ITR sequences. In another embodiment, the transgene can be flanked by inverted terminal repeat sequences from two different parvoviruses. For example, the 5′ ITR can be derived from AAV-2 and the 3′ ITR can be derived from AAV-5, as long as at least one ITR comprises a packaging sequence required to package the chimeric capsid. In one embodiment, the chimeric capsid is produced with one ITR sequence from a AAV-2 and the second ITR from a parvovirus selected from the group consisting of AAV-1, AAV-2, AAV-3, AAV-4, AAV-5, and AAV-6. In a preferred embodiment, the ITR sequences are form AAV-2. In another embodiment, the transgene may also be flanked with an ITR sequence from a parvovirus and an ITR sequence from a virus. For example, the 5′ ITR can be derived from AAV-2 and the 3′ ITR can be derived from an adenovirus as long as at least one ITR comprises a packaging sequence to package the chimeric capsid.

The ITR sequences for AAV-2 are described, for example by Kotin et al. (1994) Human Gene Therapy 5:793-801; Berns “Parvoviridae and their Replication” in Fundamental Virology, 2nd Edition, (B. N. Fields and D. M. Knipe, eds.) The skilled artisan will appreciate that AAV ITR's can be modified using standard molecular biology techniques. Accordingly, AAV ITRs used in the vectors of the invention need not have a wild-type nucleotide sequence, and may be altered, e.g., by the insertion, deletion or substitution of nucleotides. The ITR's flanking the transgene need not necessarily be identical or derived from the same AAV serotype or isolate, so long as the ITR's function as intended, i.e., to allow for excision and replication of the bounded nucleotide sequence of interest when AAV rep gene products are present in the cell.

The recombinant vector can be constructed by directly inserting the transgene into an AAV genome which has had the major AAV open reading frames (“ORFs”) excised therefrom. Other portions of the AAV genome can also be deleted, as long as a sufficient portion of the ITRs remain to allow for replication and packaging functions. These constructs can be designed using techniques well known in the art. (See, e.g., Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling et al. (1994) Gene Therapy 1:165-169; and Zhou et al. (1994) J. Exp. Med. 179:1867-1875).

Deletion or replacement of the AAV genome, e.g., the capsid region of the AAV-2, results in an AAV-2 nucleic acid which is incapable of encapsidating itself. The chimeric capsid proteins can be provided using a nucleic acid construct that encodes the chimeric capsid proteins. The chimeric capsid proteins are provided in one or more expression vector(s) which are introduced into a host cell along with the AAV-2 nucleic acid.

Plasmid expression vectors can typically be designed and constructed such that they contain a transgene encoding a protein or a portion of a protein necessary for encapsidation of the recombinant AAV-2 nucleic acid i.e., the chimeric capsid proteins. Generally, construction of such plasmids can be performed using standard methods, such as those described in Sambrook, J. et al. Molecular Cloning: A Laboratory Manual, 2nd edition (CSHL Press, Cold Spring Harbor, N. Y. 1989). The expression vector which expresses the chimeric capsid protein for encapsidation of the AAV-2 nucleic acid is constructed by first positioning the transgene to be inserted (e.g., VP1, VP2 or VP3) after a DNA sequence know to act as a promoter when introduced into cells. The transgene is typically positioned downstream (3′) from the promoter sequence. Stratagene Cloning Systems (LaJolla, Calif.), and Clontech (Palo Alto, Calif.)

The conditions under which plasmid expression vectors are introduced into a host cell vary depending on certain factors. These factors include, for example, the size of the nucleic acid of the plasmid, the type of host cell, and the desired efficiency of transfection. There are several methods of introducing the recombinant nucleic acid into the host cells which are well-known and commonly employed by those of ordinary skill in the art. These transfection methods include, for example, calcium phosphate-mediated uptake of nucleic acids by a host cell and DEAE-dextran facilitated uptake of nucleic acid by a host cell. Alternatively, nucleic acids can be introduced into cells through electroporation, (Neumann et al. (1982) EMBO J. 1:841-845), which is the transport of nucleic acids directly across a cell membrane by means of an electric current or through the use of cationic liposomes (e.g. lipofection, Gibco/BRL (Gaithersburg, Md.)). The methods that are most efficient in each case are typically determined empirically upon consideration of the above factors.

As with plasmid expression vectors, viral expression vectors can be designed and constructed such that they contain a foreign gene encoding a foreign protein or fragment thereof and the regulatory elements necessary for expressing the foreign protein. Examples of such viruses include retroviruses, adenoviruses and herpesvirus.

The entry of viral expression vectors into host cells generally requires addition of the virus to the host cell media followed by an incubation period during which the virus enters the cell. Incubation conditions, such as the length of incubation and the temperature under which the incubation is carried out, vary depending on the type of host cell and the type of viral expression vector used. Determination of these parameters is well known to those having ordinary skill in the art. In most cases, the incubation conditions for the infection of cells with viruses typically involves the incubation of the virus in serum-free medium (minimal volume) with the tissue culture cells at 30° C.for a minimum of thirty minutes. For some viruses, such as retroviruses, a compound to facilitate the interaction of the virus with the host cell is added.

Recombinant AAV vectors can be packaged into particles by co-transfection of cells with a plasmid bearing the AAV replication and/or chimeric cap genes. The replication and cap genes encode replication proteins or chimeric capsid proteins, respectively and mediate replication and genomic integration of AAV sequence, as well as packaging and formation of AAV particles (Samulski (1993) Current Opinion in Genetics and Development 3:74-80; Muzyczka, (1992) Curr. Top. Microbiol. Immunol. 158:97-129). Vectors without the rep gene appear to replicate and integrate at random sites in the host cell genome, while expression of Rep proteins Rep 68 and Rep 78, can mediate genomic integration into a well-defined locus on human chromosome 19 (Kotin, et al., Proc. Natl. Acad. Sci. USA 87:2211-2215 (1990); Samulski, et al., (1991) EMBO J 10:3941-3950; Giraud, et al., (1994) Proc. Natl. Acad. Sci. USA 91:10039-10043; Weitzman et al., (1994) Proc. Natl. Acad. Sci. USA 91:5808-5812). The plasmid bearing the cap genes can encode a chimeric capsid comprising a cap gene from a parvovirus, e.g., AAV-1, AAV-2, AAV-3, AAV-4, AAV-5 and AAV-6 or a portion thereof, or a virus, e.g., herpesvirus, adenovirus, lentivirus, retrovirus, Epstein-Barr virus and vaccinia virus. In a preferred embodiment, the chimeric capsid coat comprises the native amino acid sequence of the VP1 is derived from the AAV-2 serotype and the non-native amino acid sequence of VP2 and VP3 are derived from the AAV-5 serotype.

Suitable host cells for producing particles comprising the chimeric capsids include, but are not limited to, microorganisms, yeast cells, insect cells, and mammalian cells, that can be, or have been, used as recipients of a exogenous nucleic acid molecule.

Cells from the stable human cell line, 293 (readily available through, e.g., the ATCC under Accession No. ATCC CRL1573) are preferred in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral Ela and Elb genes (Aiello et al. (1979) Virology 94:460). The 293 cell line is readily transfected, and provides a particularly convenient platform in which to produce particles.

In one embodiment, the chimeric capsid can be produced in a suitable host cell and the chimeric capsid can be used as a delivery vehicle for an operatively linked transgene.

Standard methods of infectivity known to the skilled artisan can be used to test for the alter tropism (See e.g., Grimm et al. (1998) Hum Gene Ther 10: 2745-60). For example, efficiency of entry can be quantitated by introducing a recombinant vector with a chimeric capsid into the wild type AAV vector and monitoring transduction as a function of multiplicity of infection (MOI). A reduced MOI of the recombinant vector comprising chimeric capsid compared to a recombinant vector with a wild type capsid indicates a more efficient vector. For example, requires fewer AAV-5 particles to get one transduced cell in a target organ, e.g., brain, than that of AAV-2.

II Recombinant Vectors Comprising Chimeric Capsids Constructed From Parvovirus and a Virus

Alternatively, the recombinant vector of the invention can be a vector comprising a chimeric capsid containing amino acid sequences from a parvovirus, and a non-native amino acid sequence from a virus. Examples of a suitable virus include, but are not limited to, AAV-1, AAV-2, AAV-3, AAV-4, AAV-5, and AAV-6. Examples of a suitable virus include, but are not limited to, herpesvirus, adenovirus, lentivirus, retrovirus, Epstein-Barr virus and vaccinia virus. The recombinant vector with a chimeric capsid can have an altered tropism that allows the capsid coat to bind to the surface of cell types with a higher affinity than a recombinant vector with a wild type capsid. Alternatively, the modified tropism prevents the capsid from targeting particular cell types.

The skilled artisan can appreciate there are numerous viruses that can comprise capsid proteins which can be used to construct the recombinant vector with the chimeric capsid. For example, the herpesviruses is a large double stranded DNA viruses consisting of an icosahedral capsid surrounded by an envelope. The group has been classified as alpha, beta and gamma herpesviruses on the basis of genome structure and biological properties (See e.g., Roizman. et al. (1981) Int. virology 16, 201-217). The herpes particle constitutes over 30 different proteins which are assembled within the host cell. About 6-8 are used in the capsid.

The herpes simplex virus 1 (HSV-1) genome specifies an abundant capsid protein complex which in denaturing gels forms multiple bands due to different molecular weights of the component proteins. Details of the HSV-1 capsid have been well documented, see for example, Davison et al. (1992) J. Gen. Virol. 73:2709-2713; Gibson et al. (1972) J. Virol. 10: 1044-1052; and Newcomb et al., (1991) J. Virol., 65:613-620). Several herpesvirus sequences are available from GenBank.

The human adenovirus is comprised of a linear 36 kilobase double-stranded DNA genome, which is divided into 100 map units, each of which is 360 base pair in length. The DNA contains short inverted terminal repeats (ITR) at each end of the genome that are required for viral DNA replication. The gene products are organized into early (E1 through E4) and late (L1 through L5) regions, based on expression before or after the initiation of viral DNA synthesis (See, e.g., Horwitz, Virology, 2d edit., ed. B. N. Fields, Raven Press, Ltd. New York (1990)).

The adenovirus capsid has been well characterized and nucleic acid molecules of various adenoviruses are available in GenBank. Adenovirus interacts with eukaryotic cells by virtue of specific receptor recognition by domains in the knob portion of the fiber protein which protrude from each of the twelve vertices of the icosahedral capsid (See e.g., Henry et al. (1994) J. Virol. 68:5239-5246; Stevenson et al. (1995) J. Virol. 69:2850-2857; and Louis et al. (1994) J. Virol. 68:4104-4106). These or other regions of the adenovirus capsid may be used to construct the chimeric capsid of the invention. Nucleic acid sequences of many lentivirus, retrovirus types are available from GenBank.

III Administration of Recombinant Vectors Comprising Chimeric Capsids

Administration of the recombinant vector comprising a chimeric capsid to the cell can be accomplished by standard methods in the art. Preferably, the vector is packaged into a particle and the particle is added to the cells at the appropriate multiplicity of infection. The modified tropism of the recombinant vector allows the chimeric capsid to interact with an attachment site on a cell surface that a wild type capsid fails to interact with, for example, the AAV-2 has a poor tropism for human myeloid stem cells or cells of lymphocyte lineage. However, a recombinant vector with a chimeric capsid comprising non-native capsid proteins from different member of the parvovirus family can confer the ability to AAV-2 to interact with human myeloid stem cells. Alternatively, the modified tropism can prevent the chimeric capsid from interacting with a particular cell type, to thereby selectively target desired cell types.

Administration of the recombinant vector comprising the chimeric capsid to the cell can be by any means, including contacting the recombinant vector with the cell. For such in vitro method, the vector can be administered to the cell by standard transduction methods. (See e.g., Sambrook, Supra.) The cells being transduced can be derived from a human, and other mammals such as primates, horse, sheep, goat, pig, dog, rat, and mouse. Cell types and tissues that can be targeted include, but are not limited to, adipocytes, adenocyte, adrenal cortex, amnion, aorta, ascites, astrocyte, bladder, bone, bone marrow, brain, breast, bronchus, cardiac muscle, cecum, cervix, chorion, colon, conjunctiva, connective tissue, cornea, dermis, duodenum, endometrium, endothelium, epithelial tissue, epidermis, esophagus, eye, fascia, fibroblasts, foreskin, gastric, glial cells, glioblast, gonad, hepatic cells, histocyte, ileum, intestine, small intestine, jejumim, keratinocytes, kidney, larynx, leukocytes, lipocyte, liver, lung, lymph node, lymphoblast, lymphocytes, macrophages, mammary alveolar nodule, mammary gland, mastocyte, maxilla, melanocytes, monocytes, mouth, myelin, nervous tissue, neuroblast, neurons, neuroglia, osteoblasts, osteogenic cells, ovary, palate, pancreas, papilloma, peritoneum, pituicytes, pharynx, placenta, plasma cells, pleura, prostate, rectum, salivary gland, skeletal muscle, skin, smooth muscle, somatic, spleen, squamous, stomach, submandibular gland, submaxillary gland, synoviocytes, testis, thymus, thyroid, trabeculae, trachea, turbinate, umbilical cord, ureter, and uterus.

The recombinant vectors comprising the chimeric capsid can be incorporated into pharmaceutical compositions suitable for administration to a subject. Typically, the pharmaceutical composition comprises the recombinant vectors of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The recombinant vectors of the invention can be incorporated into a pharmaceutical composition suitable for parenteral administration. Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antigen, antibody or antibody portion) in the required amount in an appropriate solvent with one or a combination of ingredients enumerated above, as required, followed by filtered sterilization.

Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The pharmaceutical compositions of the invention may include a “therapeutically effective amount” or a “prophylactically effective amount” of the recombinant vector. A “therapeutically effective amount” refers to an amount effective, at dosages and for periods of time necessary, to achieve the desired therapeutic result. A therapeutically effective amount of the recombinant vector may vary according to factors such as the disease state, age, sex, and weight of the individual and the ability of the vector to elicit a desired response in the individual. A therapeutically effective amount is also one in which any toxic or detrimental effects of the recombinant vector is outweighed by the therapeutically beneficial effects. A “prophylactically effective amount” refers to an amount effective, at dosages and for periods of time necessary, to achieve the desired prophylactic result.

IV. Therapeutic Uses of Recombinant Vectors with Chimeric Capsids

The recombinant vectors with the chimeric capsids of the invention offer the advantage over current vector systems for gene delivery into cells. The recombinant vectors of the invention, due to their modified tropism, can efficiently and safely deliver transgenes to cells that are not normally targeted by vectors with a wild type capsid. The recombinant vectors of the invention may also be used to selectively target desired cell types, while excluded of the cell types based on the modified tropism. The recombinant vector with a chimeric capsid can comprise a transgene sequence that is associated with a disease or a disorder such that expression of the transgene would result in amelioration of the disease or disorder. There are a number of inherited neurological and metabolic diseases in which defective genes are known and have been cloned. For example, in humans, genes for defective enzymes have been identified for lysosomal storage disease, Lesch-Nyhan syndrome, amyloid polyneuropathy, Alzheimer amyloid, Duchenne's muscular dystrophy, for example. In addition, a number of other genetic diseases and disorders in which the gene associated with the disorder has been cloned or identified include diseases the of blood, such as, sickle-cell anemia, clotting disorders and thalassemias, cystic fibrosis, diabetes, disorders of the liver and lung, diseases associated with hormone deficiencies. Gene therapy could also be used to treat retinoblastoma, and various types of neoplastic cells which include tumors, neoplasms carcinomas, sarcomas, leukemias, lymphoma, and the like. Of particular interest are the central nervous system tumors. These include astrocytomas, oligodendrogliomas, meningiomas, neurofibromas, ependymomas, Schwannomas, neurofibrosarcomas, glioblastomas, and the like. For these disease and disorders, gene therapy could be used to bring a normal gene into affected tissues or replace a defective gene for replacement therapy.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

### EXAMPLES

### Example 1

Construction of a Chimeric Vector

A chimeric vector designated pHyb25 was constructed using standard molecule biology procedures. The AAV5 capsid sequence and the AAV2 rep sequence were PCR amplified separately. The AAV5 capsid gene was amplified using primers that corresponded with nucleotide positions 2207-2227 in AAV genome 5′-caataaatgatttaaatcaggtatgtcttttgttgatcaccc-3′ (SEQ ID NO: 3) and nucleotide positions 4350-4381 in AAV genome 5′-gatgttgtaagctgttattcattgaatgacc-3′ (SEQ ID NO: 4). The partial AAV2 rep sequence was amplified using primers that corresponded with nucleotide positions 2182-2202 in AAV2 genome 5′-gggtgatcaacaaaagacatacctgatttaaatcatttattg-3′ (SEQ ID NO: 5) and nucleotide positions 455-486 in AAV2 genome 5′-gattgagcaggcacccctgaccgtggccg-3′ (SEQ ID NO:6). The subsequent PCR products were linked together by PCR amplification using primers 5′-gatgttgtaagctgttattcattgaatgacc-3′ (SEQ ID NO: 4) and 5′ -gattgagcaggcacccctgaccgtggccg-3′ (SEQ ID NO: 6). After the PCR reaction, the PCR product was digested with HindlIl and the larger fragment was cloned into p5E18 at the HindIIl and Smal cloning sites as described by Xiao et al. (1999) J. Virol. 73:3994-4003. The resulting plasmid is pHyb25, a recombinant chimeric adeno-associated virus with an AAV5 capsid and AAV2 rep sequences.

### Example 2

In-vitro Infectivity of Chimeric Vector

To test the in-vitro infectivity of the recombinant chimeric plasmid, pHyb25 was cotransfected into 293 cells along with a vector plasmid with a reporter gene such as green fluorescent protein (GFP) or lacZ. The cells were infected with adenovirus at moi 5 and harvested 48 hours post adenovirus infection. The infectious particle were tested for GFP and lacZ expression in 293 cells using cell lysate from the above preparation. At MOIs of 10-1000, robust expression was seen with the recombinant chimeric pHyb25 virus.

A direct comparison was made between the recombinant chimeric Hyb25 virus and an identical expression cassette packaged into AAV-2. At all MOIs transduction efficiencies were significantly greater for AAV-5 compared to AAV-2. The data demonstrated that for a MOI (based on genomic particle titer) of 100, transduction efficiencies ranged from 80-100% for AAV-5 chimeric capsid vector, whereas with AAV-2 transduction efficiencies were consistently less ranging from 10-30%.

### Example 3

In vivo Effect of the Chimeric Vector

To test the in vivo effect of the chimeric vector, the chimeric AAV-5 vector was prepared by transfection using mini-adenovirus plasmid, pHyb25 and vector plasmid with GFP as reporter gene. The viruses were purified by CsCl gradient. 2 ml of a 1 ml genomic particle stock was injected into cortex, hippocampus and striatum of rats (n=2) per area for both AAV-2 and the chimeric AAV-5. Semi-quantitative analysis of gene expression showed a 2-10 fold increase in the number of GFP fluorescent cells with the chimeric AAV-5 vector. Moreover, >10% of transduced cells were non-neuronal including glial cells (GFAP positive) with the chimeric AAV-5 vector, whereas over 98% of cells transduced by AAV-2 were neurons.

This data collectively demonstrates that the chimeric vector had both altered tropism and increased transduction efficiency compared to the parent AAV-2 vector.

What is claimed is:

1. A recombinant viral vector comprising:
a chimeric capsid having at least one non-native amino acid sequence, wherein the non-native amino acid sequence is derived from a capsid protein domain of a parvovirus, a virus, or a combination thereof, and wherein the chimeric capsid is capable of binding to an attachment site present on a cell surface; and
a transgene flanked 5′ and 3′ by inverted terminal repeat sequences, wherein the inverted terminal repeat sequences are derived from a parvovirus, a virus, or a combination thereof, and wherein at least one inverted terminal repeat sequence comprises a packaging signal that allows assembly of the chimeric capsid.

2. The recombinant viral vector of claim 1, wherein the chimeric capsid has a modified tropism.

3. The recombinant viral vector of claim 2, wherein the chimeric capsid with a modified tropism permits binding of the viral vector to an attachment site on a cell surface with higher affinity than a corresponding viral vector with a wild type capsid.

4. The recombinant viral vector of claim 1, wherein the parvovirus selected from the group consisting of AAV-1, AAV-2, AAV-3, AAV-4, AAV-5 and AAV-6.

5. The recombinant viral vector of claim 4, wherein the parvovirus comprises a capsid protein with viral protein domain selected from the group consisting of VP1, VP2 and VP3.

6. The recombinant viral vector of claim 1, wherein the non-native amino acid sequence is a combination of amino acid sequences derived from one or more parvoviruses selected from the group consisting of AAV-1, AAV-2, AAV-3, AAV-4, AAV-5 and AAV-6.

7. The recombinant viral vector of claim 6, wherein the non-native amino acid sequence is a combination of an amino acid sequence derived from AAV-2 and an amino acid sequence derived from AAV-5.

8. The recombinant viral vector of claim 1, wherein the non-native amino acid sequence is derived from a virus.

9. The recombinant viral vector of claim 8, wherein the virus is selected from the group consisting of herpesvirus, adenovirus, lentivirus, retrovirus, Epstein-Barr virus and vaccinia virus.

10. The recombinant viral vector of claim 1, wherein the non-native amino acid sequence is a combination of at least one amino acid sequence derived from a parvovirus selected from the group consisting of AAV-1, AAV-2, AAV-3, AAV-4, AAV-5 and AAV-6, and at least one amino acid sequence derived from a virus selected from the group consisting of herpesvirus, adenovirus, lentivirus, retrovirus, Epstein-Barr virus and vaccinia virus.

11. The recombinant viral vector of claim 1, wherein the inverted terminal repeat sequences are each derived from a parvovirus selected from the group consisting of AAV-1, AAV-2, AAV-3, AAV-4, AAV-5 and AAV-6.

12. The recombinant viral vector of claim 1, wherein the inverted terminal repeat sequences are each derived from a viruses selected from the group consisting of herpesvirus, adenovirus, lentivirus, retrovirus, Epstein-Barr virus and vaccinia virus.

13. The recombinant viral vector of claim 1, wherein the inverted terminal repeat sequences are a combination of at least one inverted terminal repeat sequence derived from a parvovirus selected from the group consisting of AAV-1, AAV-2, AAV-3, AAV-4, AAV-5 and AAV-6, and at least one inverted terminal repeat sequence derived from a virus selected from the group consisting of herpesvirus, adenovirus, lentivirus, retrovirus, Epstein-Barr virus and vaccinia virus.

14. The recombinant viral vector of claim 1, wherein the transgene is selected from the group consisting of an RNA molecule, a DNA molecule, and a synthetic DNA molecule.

15. A recombinant AAV-2 vector comprising:
a chimeric capsid having at least one native AAV-2 amino acid sequence, and at least one non-native amino acid sequence derived from a parvovirus other than AAV-2, wherein the chimeric capsid is capable of binding to an attachment site present on a cell surface; and
a transgene flanked 5′ and 3′ by a first inverted terminal repeat sequence derived from AAV-2 and a second inverted terminal repeat sequence derived from a parvovirus.

16. The recombinant AAV-2 vector of claim 15, wherein the chimeric capsid has a modified tropism.

17. The recombinant AAV-2 vector of claim 16, wherein the chimeric capsid with a modified tropism permits binding of the AAV-2 vector to an attachment site on a cell surface with higher affinity than that exhibited by a corresponding AAV-2 vector with a wild type AAV-2 capsid.

18. The recombinant AAV-2 vector of claim 15, wherein the amino acid sequence derived from AAV-2 comprises a viral protein domain selected from the group consisting of VP1, VP2 and VP3.

19. The recombinant AAV-2 vector of claim 15, wherein the non-native amino acid sequence is derived from a parvovirus selected from the group consisting of AAV-1, AAV-3, AAV-5 and AAV-6.

20. The recombinant AAV-2 vector of claim 19, wherein the non-native amino acid sequence of the parvovirus comprises a viral protein domain selected from the group consisting of VP1, VP2 and VP3.

21. The recombinant AAV-2 vector of claim 15, wherein the chimeric capsid comprises a native amino acid sequence derived from the VP1 domain of AAV-2 and, wherein the non-native amino acid sequence comprises a VP2 domain and a VP3 domain derived from AAV-5.

22. The recombinant AAV-2 vector of claim 15, wherein the second inverted terminal repeat sequence derived from a parvovirus is selected from the group consisting of AAV-1, AAV-2, AAV-3, AAV-4, AAV-5 and AAV-6.

23. The recombinant AAV-2 vector of claim 15, wherein the transgene is selected from the group consisting of an RNA molecule, a DNA molecule, and a synthetic DNA molecule.

24. A recombinant AAV-2 vector comprising:
a chimeric capsid having at least one native AAV-2 amino acid sequence and at least one non-native amino acid sequence derived from a virus, wherein the chimeric capsid is capable of binding to an attachment site present on a cell surface; and
a transgene flanked 5′ and 3′ by a first inverted terminal repeat sequence derived from AAV-2 and a second inverted terminal repeat sequence derived from a parvovirus.

25. The recombinant AAV-2 vector of claim 24, wherein the chimeric capsid has a modified tropism.

26. The recombinant AAV-2 vector of claim 25, wherein the chimeric capsid with a modified tropism permits binding of the AAV-2 vector to an attachment site on a cell surface with higher affinity than a corresponding AAV-2 vector with a wild type capsid.

27. The recombinant AAV-2 vector of claim 24, wherein the amino acid sequence derived from AAV-2 comprises a viral protein domain selected from the group consisting of VP1, VP2 and VP3.

28. The recombinant AAV-2 vector of claim 24, wherein the non-native amino acid sequence is derived from a virus selected from the group consisting of herpesvirus, adenovirus, lentivirus, retrovirus, Epstein-Barr virus and vaccinia virus.

29. The recombinant AAV-2 vector of claim 24, wherein the second inverted terminal repeat sequence is derived from a parvovirus selected from the group consisting of AAV-1, AAV-3, AAV-4, AAV-5 and AAV-6.

30. The recombinant AAV-2 vector of claim 24, wherein the transgene is selected from the group consisting of an RNA molecule, a DNA molecule, and a synthetic DNA molecule.

31. A recombinant AAV-2 vector comprising:
a chimeric capsid having at least one native AAV-2 amino acid sequence, and at least one non-native amino acid sequence derived from a virus, wherein the chimeric capsid is capable of binding to an attachment site present on a cell surface; and
a transgene flanked by a first inverted terminal repeat sequence derived from AAV-2 and a second inverted terminal repeat sequence derived from a virus.

32. The recombinant AAV-2 vector of claim 31, wherein the chimeric capsid has a modified tropism.

33. The recombinant AAV-2 vector of claim 32, wherein the chimeric capsid with a modified tropism permits binding of the AAV-2 vector to an attachment site on a cell surface with higher affinity than a corresponding AAV-2 vector with a wild type capsid.

34. The recombinant AAV-2 vector of claim 31, wherein the amino acid sequence derived from AAV-2 comprises a viral protein domain selected from the group consisting of VP1, VP2 and VP3.

35. The recombinant AAV-2 vector of claim 31, wherein the non-native amino acid sequence is derived from a virus selected from the group consisting of herpesvirus, adenovirus, lentivirus, retrovirus, Epstein-Barr virus and vaccinia virus.

36. The recombinant AAV-2 vector of claim 31, wherein the second terminal repeat sequence is derived from a virus selected from the group consisting of herpesvirus, adenovirus, lentivirus, retrovirus, Epstein-Barr virus and vaccinia virus.

37. The recombinant AAV-2 vector of claim 31, wherein the transgene is selected from the group consisting of an RNA molecule, a DNA molecule, and a synthetic DNA molecule.

38. A chimeric capsid vehicle comprising a native AAV-2 amino acid sequence and at least one non-native amino acid sequence derived from a capsid protein of a parvovirus other than AAV-2, covalently linked to a transgene.

39. The chimeric capsid vehicle of claim 38 wherein the chimeric capsid has a modified tropism.

40. The chimeric capsid vehicle of claim 39, wherein the chimeric capsid with a modified tropism permits binding of the chimeric capsid to an attachment site on a cell surface with higher affinity than a corresponding wild type capsid vehicle.

41. The chimeric capsid vehicle of claim 38, wherein the amino acid sequence derived from AAV-2 comprises a viral protein domain selected from the group consisting of VP 1, VP2 and VP3.

42. The chimeric capsid vehicle of claim 38, wherein the non-native amino acid sequence is derived from a parvovirus selected from the group consisting of AAV-1, AAV-3, AAV-5 and AAV-6.

43. The chimeric capsid vehicle of claim 38, wherein the transgene is selected from the group consisting of an RNA molecule, a DNA molecule, and a synthetic DNA molecule.

44. A chimeric capsid vehicle comprising a native AAV-2 amino acid sequence and at least one non-native amino acid derived from a capsid protein of a virus, covalently linked to a transgene.

45. The chimeric capsid vehicle of claim 44 wherein the chimeric capsid has a modified tropism.

46. The chimeric capsid vehicle of claim 45, wherein the chimeric capsid with a modified tropism permits binding of the chimeric capsid to an attachment site on a cell surface with higher affinity than a corresponding wild type capsid vehicle.

47. The chimeric capsid vehicle of claim 44, wherein the amino acid sequence derived from AAV-2 comprises a viral protein domain selected from the group consisting of VP1, VP2 and VP3.

48. The chimeric capsid vehicle of claim 44, wherein the non-native amino acid sequence is derived from a virus selected from the group consisting of herpesvirus, adenovirus, lentivirus, retrovirus, Epstein-Barr virus and vaccinia virus.

49. The chimeric capsid vehicle of claim 44, wherein the transgene is selected from the group consisting of an RNA molecule, a DNA molecule, and a synthetic DNA molecule.

50. A method for modifying the tropism of a recombinant AAV-2 vector comprising:
replacing at least a portion of a native amino acid sequence of an AAV-2 capsid protein with a non-native amino acid sequence derived from a capsid protein of a parvovirus other than AAV-2; and
combining the capsid proteins under conditions for assembly to produce a chimeric capsid encapsidating an AAV-2 vector, to thereby modify the tropism of an AAV-2 vector.

51. The method of claim 50, wherein the parvovirus is selected from the group consisting of AAV-1, AAV-3, AAV-5 and AAV-6.

52. A method for modifying the tropism of a recombinant AAV-2 vector comprising:
replacing at least a portion of a native amino acid sequence of an AAV-2 capsid protein with a non-native amino acid sequence derived from a capsid protein of a virus; and
combining the capsid protein under conditions for assembly, to thereby modify the tropism of an AAV-2 vector.

53. The method of claim 52, wherein the non-native amino acid sequence is derived from a virus selected from the group consisting of herpesvirus, adenovirus, lentivirus, retrovirus, Epstein-Barr virus and vaccinia virus.

54. A method for improving gene therapy in a subject with a disorder comprising:
administering a therapeutically effective amount of a recombinant vector comprising a transgene and a chimeric capsid capable of binding to an attachment site present on a cell surface;
targeting a cell that recombinant vector with a chimeric capsid can bind to with a higher affinity than the corresponding viral vector with a wild type capsid; and
expressing the transgene in a subject at a level sufficient to ameliorate the disorder, thereby improving gene therapy.

55. The method of claim 54, wherein the step of administering the recombinant vector with a chimeric capsid further comprises administering a recombinant vector comprising a chimeric capsid with at least one amino acid sequence derived from a first parvovirus and at least one amino acid sequence derived from a second parvovirus.

56. The method of claim 55, wherein the first parvovirus is selected from the group consisting of AAV-l, AAV-2, AAV-3, AAV-4, AAV-5 and AAV-6.

57. The method of claim 55, wherein the second parvovirus is selected from the group consisting of AAV-l, AAV-2, AAV-3, AAV-4, AAV-5 and AAV-6.

58. The method of claim 54, wherein the step of administering the recombinant vector with a chimeric capsid comprises administering a recombinant vector comprising a chimeric capsid with at least one amino acid sequence derived from a parvovirus and at least one amino acid sequence derived from a virus.

59. The method of claim 58, wherein the parvovirus is selected from the group consisting of AAV-1, AAV-2, AAV-3, AAV-4, AAV-5 and AAV-6.

60. The method of claim 58, wherein the virus is selected from the group consisting of herpesvirus, adenovirus, lentivirus, retrovirus, Epstein-Barr virus and vaccinia virus.

61. The method of claim 54, wherein the step of administering the recombinant vector with a chimeric capsid comprises administering a recombinant vector comprising a chimeric capsid with at least one amino acid sequence derived from AAV-2 and at least one amino acid sequence derived from a parvovirus.

62. The method of claim 61, wherein the parvovirus is selected from the group consisting of AAV-1, AAV-3, AAV-5 and AAV-6.

63. The method of claim 54, wherein the step of administering the recombinant vector with a chimeric capsid comprises administering a recombinant vector comprising a chimeric capsid with at least one amino acid sequence derived from AAV-2 and at least one amino acid sequence derived from a virus.

64. The method of claim 63, wherein the virus is selected from the group consisting of herpesvirus, adenovirus, lentivirus, retrovirus, Epstein-Barr virus and vaccinia virus.

65. A method for increasing the efficiency of entry into a cell using a recombinant viral vector with a chimeric capsid comprising:
producing a chimeric capsid encapsidating a viral vector, wherein the chimeric capsid has a modified tropism; and
contacting a cell with the recombinant viral vector having a chimeric capsid such that the chimeric capsid binds to an attachment site on the cell surface and permits the vector to enter the cell more efficiently that a viral vector comprising a wild type capsid.

66. The method of claim 65, wherein the step of producing a chimeric capsid encapsidating a viral vector comprises producing a chimeric capsid with at least one amino acid sequence derived from a first parvovirus and at least one amino acid sequence derived from a second parvovirus.

67. The method of claim 66, wherein the first parvovirus is selected from the group consisting of AAV-1, AAV-2, AAV-3, AAV-4, AAV-5 and AAV-6.

68. The method of claim 66, wherein the second parvovirus is selected from the group consisting of AAV-1, AAV-2, AAV-3, AAV-4, AAV-5 and AAV-6.

69. The method of claim 65, wherein the step of producing a chimeric capsid encapsidating a viral vector comprises producing a chimeric capsid with at least one amino acid sequence derived from a parvovirus and at least one amino acid sequence derived from virus.

70. The method of claim 69, wherein the parvovirus is selected from the group consisting of AAV-1, AAV-2, AAV-3, AAV-4, AAV-5 and AAV-6.

71. The method of claim 69, wherein the virus is selected from the group consisting of herpesvirus, adenovirus, lentivirus, retrovirus, Epstein-Barr virus and vaccinia virus.

72. The method of claim 65, wherein the step of producing a chimeric capsid encapsidating a viral vector comprises producing a chimeric capsid with at least one amino acid sequence derived from AAV-2 and at least one amino acid sequence derived from a parvovirus.

73. The method of claim 72, wherein the parvovirus is selected from the group consisting of AAV-1, AAV-3, AAV-5 and AAV-6.

74. The method of claim 65, wherein the step of producing a chimeric capsid encapsidating a viral vector comprises producing a chimeric capsid with at least one amino acid sequence derived from AAV-2 and at least one amino acid sequence derived from a virus.

75. The method of claim 74, wherein the virus is selected from the group consisting of herpesvirus, adenovirus, lentivirus, retrovirus, Epstein-Barr virus and vaccinia virus.

76. A method of making a recombinant particle with a chimeric capsid comprising:
providing a first construct comprising a transgene flanked 5′ and 3′ with inverted terminal repeat sequences, wherein at least one invented terminal repeat sequence comprises a packaging signal, and a second construct comprising a nucleic acid sequence encoding a chimeric capsid; and
contacting a population of cells with the first and second constructs, such that the population of cells allows assembly of a recombinant particle, to thereby produce a recombinant particle with a chimeric capsid.

77. The method of claim 76, wherein the first construct comprises inverted terminal repeat sequences derived from one or more parvoviruses selected from the group consisting of AAV-1, AAV-2, AAV-3, AAV-4, AAV-5 and AAV-6.

78. The method of claim 76, wherein the first construct comprises inverted terminal repeat sequences derived from AAV-2.

79. The method of claim 76, wherein the second construct further comprises a nucleic acid sequence encoding a chimeric capsid of any one of claims 1, 15, 24 or 31.

80. The method of claim 76, wherein the step of contacting the population of cells further comprises contacting a population of 293 cells.

81. A cell comprising a recombinant viral vector comprising a chimeric capsid of any of claims 1, 15, 24 or 31.

82. A pharmaceutical composition comprising a recombinant viral vector comprising
a chimeric capsid of any one of claims 1, 15, 24 or 31; and
a pharmaceutically acceptable carrier.
